(19) United States
(12) Patent Application Publication
(10) Pub. No.: US 2016/0331926 A1
(43) Pub. Date: Nov. 17, 2016

(54) FIBER BRAGG GRATING-BASED PRESSURE TRANSDUCER CATHETER

(71) Applicants: Kenneth M. Bueche, Friendswood, TX (US); Timothy Daugherty, Katy, TX (US); David Nevrla, Friendswood, TX (US); Gabriel P. Kern, Houston, TX (US)

(72) Inventors: Kenneth M. Bueche, Friendswood, TX (US); Timothy Daugherty, Katy, TX (US); David Nevrla, Friendswood, TX (US); Gabriel P. Kern, Houston, TX (US)

(73) Assignee: Millar Instruments, Houston, TX (US)

(21) Appl. No.: 15/152,115

(22) Filed: May 11, 2016

Related U.S. Application Data
(60) Provisional application No. 62/159,681, filed on May 11, 2015.

(51) Int. Cl. A61M 25/00; B29C 63/00; B29C 65/00; B29C 63/18; A61B 5/00; B29C 65/70

(52) U.S. Cl. CPC A61M 25/00; A61B 5/0084; A61B 5/7217; B29C 65/70; B29C 66/534; B29C 63/18; B29C 63/0069; A61B 2034/2061

(57) ABSTRACT

A fiber optic pressure-based transducer catheter is capable of measuring pressure and temperature in the environment in which it is deployed. The pressure sensor embedded in the distal end of the catheter can be realized via optical Fiber Bragg Grating (FBG) technology.

### FIELD

The disclosure relates generally to catheters. The disclosure relates specifically to fiber optic catheters.

### BACKGROUND

Current catheters are able to measure pressure and have a small profile. However, catheters based on microelectro-mechanical systems or Fabry-Perot sensing interferometer optical systems are vulnerable to 1) drift-related problems and electro-magnetic interference and 2) bend effects, offset, or zero shifts that affect pressure readings from the sensors, respectively.

It would therefore be advantageous to have an ultra-small transducer catheter that functions within the presence of electrically noisy instruments.

### SUMMARY

An embodiment of the disclosure is a fiber optic catheter comprising a sensor housing; a diaphragm-like structure on one side of the sensor housing, wherein the diaphragm-like structure is exposed to a medium to be measured; a window located in the sensor housing; a thick-walled polymeric tubing surrounding the sensor housing; and an optical fiber with Fiber Bragg Gratings located on the diaphragm-like structure. In an embodiment, the fiber optic catheter further comprises a thin-walled polymeric tubing surrounding the thick-walled polymeric tubing. In an embodiment, the fiber optic catheter further comprises a shrink tube surrounding the thin-walled polymeric tubing. In an embodiment, the fiber optic catheter further comprises a transfer lumen. In an embodiment, the fiber optic catheter further comprises a FBG temperature sensor on the optical fiber. In an embodiment, the fiber optic catheter of claim 1 wherein the thick-walled polymeric tube is between 0.001 to 0.04 inches thick. In an embodiment, the fiber optic catheter further comprises the thin-walled polymeric tube is 0.0005-0.035 inches thick. In an embodiment, the fiber optic catheter is comprised of at least one material selected from silicone, nylon, polyimide, polyurethane, polyethylene, and Teflon/PTFE. In an embodiment, the sensor housing is comprised of at least one material selected from titanium, stainless steel, silicon, liquid crystal polymer, or other suitable rigid material. In an embodiment, the diameter of the optical fiber is between 20 μm to 125 μm.

An embodiment of the disclosure is a method of manufacturing a fiber optic catheter comprising obtaining an optical fiber with Fiber Bragg Gratings located on a diaphragm-like structure; inserting the optical fiber through a thick-walled polymeric tube; bonding the ends of the thick-walled polymeric tube to the ends of the sensor housing by reflowing polymer over the tubing and the ends of the case; placing a thin-walled polymeric tube over the thick-walled polymeric tube; inserting the thin-walled polymeric tube into a shrink tube; positioning the thin-walled polymeric tube within the shrink tube; and heating the shrink tube. In an embodiment, the thick-walled polymeric tube is between 0.001 to 0.04 inches thick. In an embodiment, the thin-walled polymeric tube is 0.0005-0.035 inches thick. In an embodiment, reflowing the polymer is around a joint and the joint is selected from the group consisting of a split shaft joint and a skive joint. In an embodiment, the proximal portion of the catheter is comprised of a high durometer material and the distal portion of the catheter is comprised of a low durometer material. In an embodiment, the catheter comprises one or more sections of radiopaque fillers. In an embodiment, a window in the thin-walled polymeric tube is positioned over the diaphragm. In an embodiment, a window over the diaphragm is cut into the thin-walled polymeric tube. In an embodiment, the fiber optic catheter is comprised of at least one material selected from silicone, nylon, polyimide, polyurethane, polyethylene, and PTFE. In an embodiment, the sensor housing is comprised of at least one material selected from titanium, stainless steel, silicon, liquid crystal polymer, and other suitable rigid material.

The foregoing has outlined rather broadly the features of the present disclosure in order that the detailed description that follows can be better understood. Additional features and advantages of the disclosure will be described hereinafter, which form the subject of the claims.

### BRIEF DESCRIPTION OF THE DRAWINGS

In order that the manner in which the above-recited and other enhancements and objects of the disclosure are obtained, a more particular description of the disclosure briefly described above will be rendered by reference to specific embodiments thereof which are illustrated in the appended drawings. Understanding that these drawings depict only typical embodiments of the disclosure and are therefore not to be considered limiting of its scope, the disclosure will be described with additional specificity and detail through the use of the accompanying drawings in which:

FIG. 1 depicts a tip design for a non-guided pressure transducer catheter;

FIG. 2 depicts a cross-sectional view (A-A) of FIG. 4;

FIG. 3 depicts a cross-sectional view (B-B) of the rapid exchange configuration of FIG. 5;

FIG. 4 depicts a distal monorail tip design for a pressure transducer catheter;

FIG. 5 depicts a rapid exchange distal configuration;

### DETAILED DESCRIPTION

The particulars shown herein are by way of example and for purposes of illustrative discussion of the preferred embodiments of the present disclosure only and are presented in the cause of providing what is believed to be the most useful and readily understood description of the principles and conceptual aspects of various embodiments of the disclosure. In this regard, no attempt is made to show structural details of the disclosure in more detail than is necessary for the fundamental understanding of the disclosure, the description taken with the drawings making apparent to those skilled in the art how the several forms of the disclosure can be embodied in practice.

The following definitions and explanations are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary 3^{rd} Edition.

As used herein, the term “durometer” means and refers to a measurement of the hardness of a material.

As used herein, the term “atraumatic” means and refers to a medical device or procedure causing a minimal tissue injury.

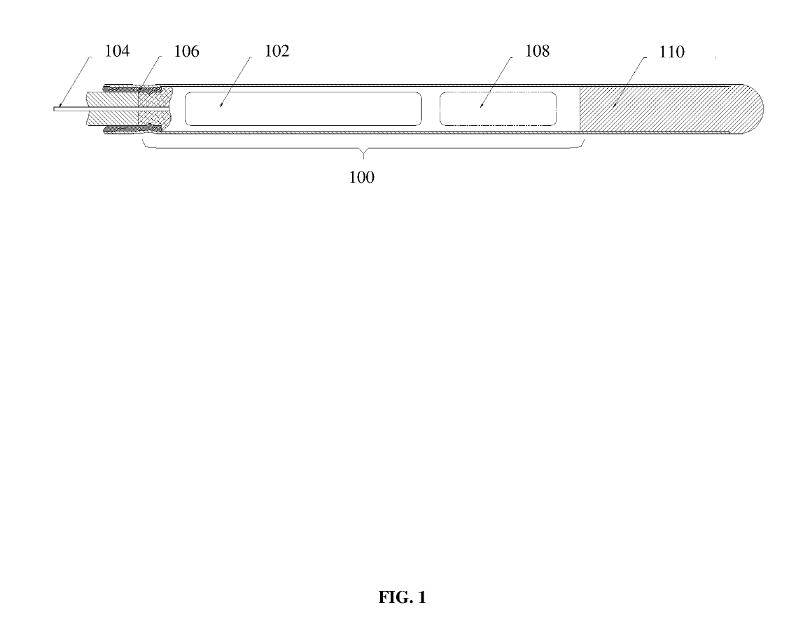

In an embodiment, a pressure transducer catheter has multiple different distal tip configurations. The pressure sensor 108, which is embedded in the distal end of the catheter, is realized via optical Fiber Bragg Gratings (FBG) technology. FBG are reflective structures in the core of an optical fiber that perturb the effective refractive index of the optical fiber. The perturbation can be periodic or aperiodic. The perturbation leads to the light being reflected in a narrow range of wavelengths. FBG technology is temperature sensitive and therefore the disclosure describes an integral temperature sensor. FIGS. 1-5. The wavelength of reflection is dependent upon the grating period, temperature, and strain. In another embodiment the pressure and temperature sensors are in separate housings to aid in manufacturability and for improved flexibility in the sensor region. The construction of the catheter as described below can be essentially the same in either embodiment. It is disclosed however, for the sake of disclosure, that both the pressure and temperature spectra can be contained within the same fiber, such that the fiber optic sensor interrogator can intercept and discern the change in spectra for both physical measurements. In an embodiment, the fiber diameter can range from 20 μm to 125 μm. In an embodiment, a 40 μm optical fiber is utilized for this design. With the spectra shift information known, an algorithm can calculate the pressure and temperature from the sensors located in the distal end of the catheter and adjust the pressure output according to the temperature compensation algorithm. The pressure and temperature reading will then be available in a variety of different means, including but not limited to analog output (including but not limited to 0-5 volts direct current (VDC)), digital output (including but not limited to RS232 (a standard for serial communication transmission of data)), and digital display.

The present disclosure discloses an ultra-small pressure transducer catheter for use within the clinical, human use environment, which includes, but is not limited to, magnetic resonance image (MRI) suites and in the presence of other electrically noisy instruments, such as electrocautery equipment. Current devices measure pressure and meet the small size catheter crossing profile requirements, but these are generally based on microelectro-mechanical systems (MEMS) or Fabry-Perot sensing interferometer optical systems. Both of these technologies have their own set of limitations. MEMS sensors are known to have drift (zero offset) related problems over time and are affected by electro-magnetic interference (EMI) such as produced by MRI machines and electrocautery equipment. The Fabry-Perot sensors are not affected by EMI but have offset or zero shifts due to changes in the curvature of the optic fiber which effect pressure readings from the sensor since these types of sensors work on the basis of quantity of reflected light.

In FBG fiber optic systems, it is not the quantity of reflected light that is measured to determine a physical event, it is a shift within the spectra of the reflected light that is observed and quantified in order to determine the physical event to which the sensor is subjected.

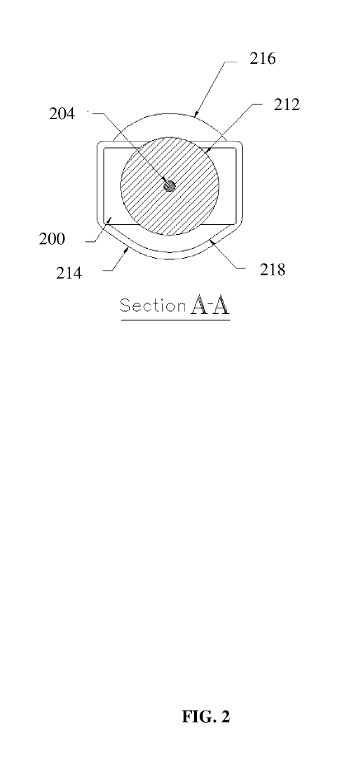

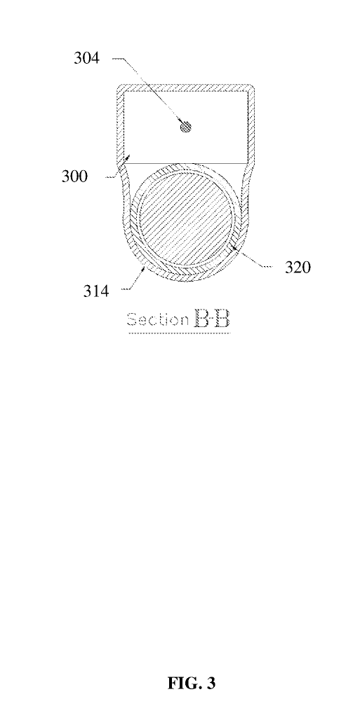

Therefore, the current catheter disclosure is one that is built up over a FBG pressure sensor which also includes an integral temperature sensor to be used for temperature compensation of the pressure reading as seen in FIG. 1. It is envisioned that the sensor housing may not have a circular cross section due to the needed manufacturing techniques of this ultra-small component or the room to afford a lumen for a guidewire, as depicted in FIGS. 2 and 3. In an embodiment, the sensor housing has a circular cross section. In an embodiment, the sensor housing does not have a circular cross section.

The sensor housing 100 can have a diaphragm-like structure 102 on one side that will need to be exposed to medium in which pressure is to be measured. The housing shall have a through hole to which the optical fiber 104 will be inserted. In an embodiment, the sensor housing 100 is made of titanium, stainless steel, silicon, liquid crystal polymer, or other suitable rigid material. In an embodiment, the sensor housing 100 is made of silicon. Optical fiber 104 is known to have relatively good tensile strength but can be compromised quite easily when bent to small radii, hence the catheter design can leverage the tensile strength to contribute to the overall strength of the catheter while protecting the fiber from small bending radii. In an embodiment, the optical fiber is comprised of glass or plastic. In an embodiment, data is transmitted through the optical fiber 104. In an embodiment, the thick-walled polymeric tube is between 0.001 to 0.04 inches thick. The ends of the thick-walled tubing can be bonded to the ends of the sensor housing by means of reflowing polymer over the tubing and the protrusion on the ends of the case. This would form the primary joint 106 between the sensor housing and the thick-walled tubing. A further embodiment can have a split shaft joint or even a skive joint which could be held together by reflowing a polymer around the joint. These types of joints help reduce the shear stress in the joint making it more robust relative to a simple butt joint. In an embodiment, any joint that reduces the shear stress can be utilized. With the cross section of the catheter body being normalized, a thin-walled polymeric tube can be placed over the previously described subassembly. This can be inserted into a shrink tube which can be positioned and transferred through a vertical oven to bring the thin-walled tubing to near melt temperature while the shrink tubing forces the material to reflow, take the shape of the underlying components, and ultimately form a protective sheathing over the catheter inner components. In an embodiment, the thin-walled polymeric tube is between 0.0005 to 0.035 inches thick. In an embodiment, the wall thickness will depend of the diameter of the finished catheter. The thin-walled tubing can either have a small window pre-formed in it which could be precisely positioned prior to the reflow operation or the window could be created after the reflow process by cutting the window with a laser such that the sensor diaphragm is exposed to the medium to be measured.

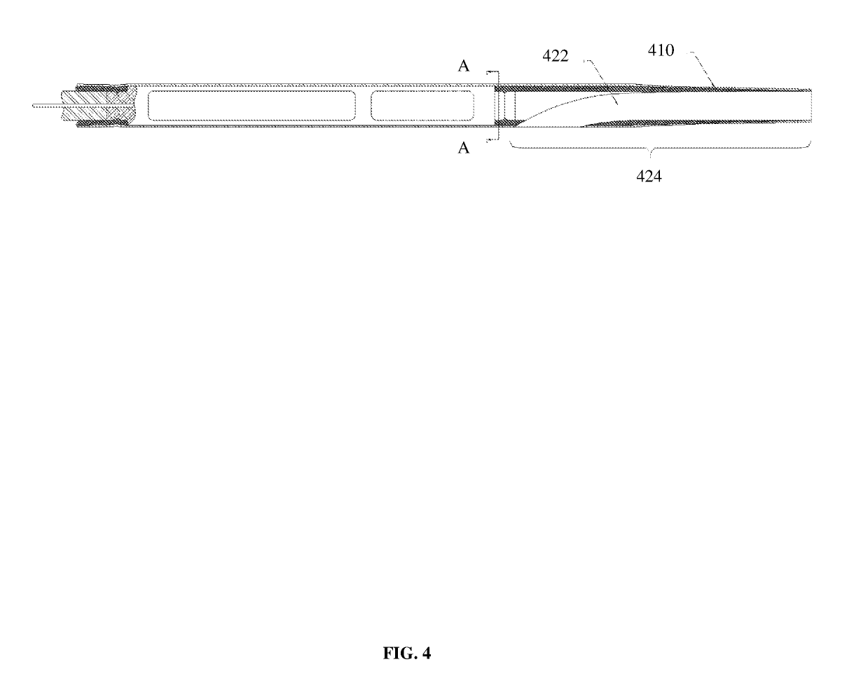

The tip configuration can take different forms. FIG. 1, depicts an atraumatic tip 110, pressure diaphragm 102, fiber 104, joint 106, and internal temperature sensor 108.In an embodiment, the device can be used in a scenario where the catheter is guided via a guide catheter to a target site in the body where pressure is being sought versus being self-guided over a guidewire. In an embodiment, the fiber is 40 μm. In an embodiment, the joint is a proximal joint. In an embodiment, in this configuration the device can be used as a pressure transducer component of a more complex medical device assembly and can provide an anchoring point for the catheter and/or an offset to ensure the sensor diaphragm is properly position relative to a port in the finished medical device. FIG. 2 depicts the cross section of such a tip configuration and also of the tip depicted on FIG. 4. FIG. 2 depicts a sensor housing 200, optical fiber 204, heavy-walled tubing 212, thin-walled tubing 214, catheter shaft 216, and sensor housing support structure 218. FIG. 4 is a drawing of a pressure transducer catheter with a distal monorail style tip where a guide wire is threaded through the tip lumen such that the catheter can be guided down the wire to the target site. FIG. 4 depicts an atraumatic tip 410, guidewire lumen 422, and distal monorail 424.

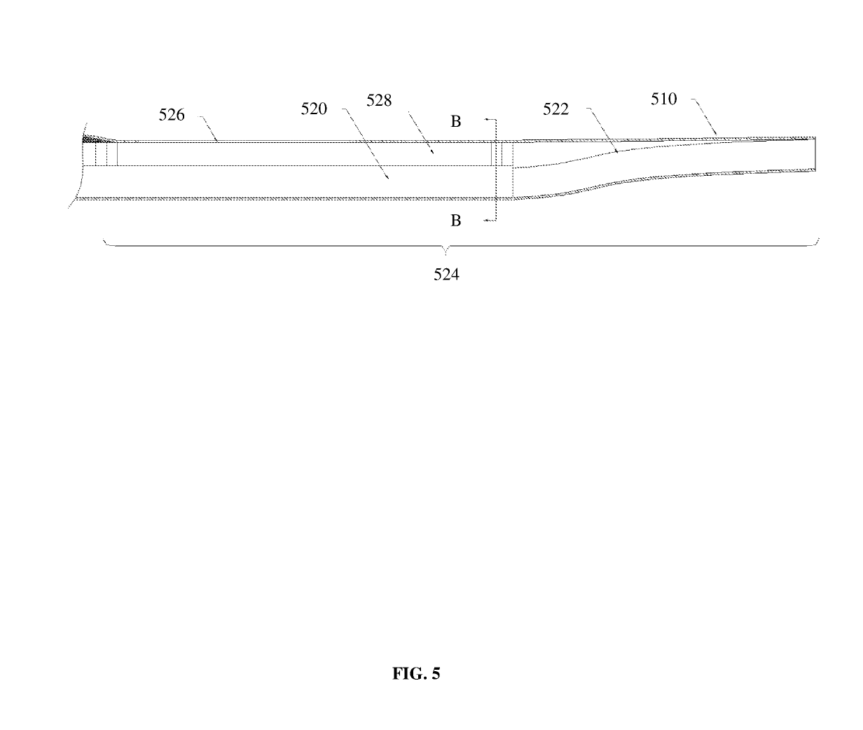

An alternate to this style of guidewire compatibility is the rapid exchange configuration, seen in FIG. 5, which allows the guidewire to pass within the catheter adjacent to the sensor housing as seen in the cross section depicted in FIG. 3. FIG. 3 depicts a sensor housing 300, optical fiber 304, thin-walled tubing 314, and a transfer lumen 320. FIG. 5 depicts an atraumatic tip 510, transfer lumen 520, guidewire lumen 522, distal monorail 524, sensor window 526, and sensor 528.

By selecting materials with different mechanical properties, including but not limited to durometer, to make the components can yield different finished catheter characteristics. In an embodiment, the durometer range of the materials is 40 Shore A to 100 Shore D. In an embodiment, a catheter is comprised of at least one of the following materials, silicone, nylon, polyimide, polyurethane, polyethylene, and Teflon/PTFE. For example, the thick-walled tubing could be pieced together with the proximal portion of the catheter being built of high durometer material followed by the distal portion being built with lower durometer materials. This type of combination would yield a catheter that has good stiffness over the majority of the catheter for optimal pushability while the remainder of the distal portion of the catheter with good trackability to make it easier for the physician to navigate to the treatment site and take pressure measurements where needed while being atraumatic to the vessels through which the catheter is traversing through.

Furthermore, the outer thin-walled tubing that forms the protective sheath of the finished device can be pieced together with sections of polymers that have been doped with radiopaque fillers to form marker bands to assist the physician in ensure the pressure window is properly positioned for more accurate pressure measurements.

In an embodiment, the proximal end of the catheter can be constructed to include a connector which can be utilized as the interface to a FBG interrogator. This connector would center the fiber relative to the interrogator. The connector can contain a means of storing catheter critical information including but not limited to calibration coefficients for the pressure and temperature sensors, and catheter identification. The storage media can communicate to the interrogator by means including but not limited to RS-232 or Bluetooth.

In an embodiment, a 2F catheter has a thin-walled polymeric tubing of 0.001 inches and a durometer of 80 Shore D. In an embodiment, the 2F catheter has a thick-walled polymeric tubing of 0.011 inches with a durometer of 70 Shore A at the distal section and a durometer of 70 Shore D at the proximal section.

In an embodiment, the catheter will be inserted into a blood vessel of patient. The pressure within the vessel can be measured and reported to the interrogator via Bluetooth.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations can be applied to the compositions and methods and in the steps or in the sequence of steps of the methods described herein without departing from the concept, spirit and scope of the disclosure. More specifically, it will be apparent that certain substances which are related can be substituted for the substances described herein while the same or similar results would be achieved. All such similar substitutes and modifications apparent to those skilled in the art are deemed to be within the spirit, scope and concept of the disclosure as defined by the appended claims.

What is claimed is:

1. A fiber optic catheter comprising
a sensor housing;
a diaphragm-like structure on one side of the sensor housing, wherein the diaphragm-like structure is exposed to a medium to be measured;
a window located in the sensor housing;
a thick-walled polymeric tubing surrounding the sensor housing; and
an optical fiber with Fiber Bragg Gratings located on the diaphragm-like structure.

2. The fiber optic catheter of claim 1 further comprising a thin-walled polymeric tubing surrounding the thick-walled polymeric tubing.

3. The fiber optic catheter of claim 2 further comprising a shrink tube surrounding the thin-walled polymeric tubing.

4. The fiber optic catheter of claim 1 further comprising a transfer lumen.

5. The fiber optic catheter of claim 1 further comprising a FBG temperature sensor on the optical fiber,

6. The fiber optic catheter of claim 1 wherein the thick-walled polymeric tube is between 0.001 to 0.04 inches thick.

7. The fiber optic catheter of claim 2 wherein the thin-walled polymeric tube is 0.0005-0.035 inches thick.

8. The fiber optic catheter of claim 1 wherein the fiber optic catheter is comprised of at least one material selected from silicone, nylon, polyimide, polyurethane, polyethylene, and Teflon/PTFE.

9. The fiber optic catheter of claim 1 wherein the sensor housing is comprised of at least one material selected from titanium, stainless steel, silicon, liquid crystal polymer, or other suitable rigid material.

10. The fiber optic catheter of claim 1 wherein the diameter of the optical fiber is between 20 μm to 125 μm.

11. A method of manufacturing a fiber optic catheter comprising
obtaining an optical fiber with Fiber Bragg Gratings located on a diaphragm-like structure;
inserting the optical fiber through a thick-walled polymeric tube;
bonding the ends of the thick-walled polymeric tube to the ends of the sensor housing by reflowing polymer over the tubing and the ends of the case;
placing a thin-walled polymeric tube over the thick-walled polymeric tube;
inserting the thin-walled polymeric tube into a shrink tube;
positioning the thin-walled polymeric tube within the shrink tube; and
heating the shrink tube.

12. The method of claim 11 wherein the thick-walled polymeric tube is between 0.001 to 0.04 inches thick.

13. The method of claim 11 wherein the thin-walled polymeric tube is 0.0005-0.035 inches thick.

14. The method of claim 11 wherein reflowing the polymer is around a joint and the joint is selected from the group consisting of a split shaft joint and a skive joint.

15. The method of claim 411 wherein the proximal portion of the catheter is comprised of a high durometer material and the distal portion of the catheter is comprised of a low durometer material.

16. The method of claim 11 wherein the catheter comprises one or more sections of radiopaque fillers.

17. The method of claim 11 wherein a window in the thin-walled polymeric tube is positioned over the diaphragm.

18. The method of claim 11 wherein a window over the diaphragm is cut into the thin-walled polymeric tube.

19. The method of claim 11 wherein the fiber optic catheter is comprised of at least one material selected from silicone, nylon, polyimide, polyurethane, polyethylene, and PTFE.

20. The method of claim 11 wherein the sensor housing is comprised of at least one material selected from titanium, stainless steel, silicon, liquid crystal polymer, and other suitable rigid material.
